(19) United States
(12) Patent Application Publication
(10) Pub. No.: US 2023/0005568 A1
(43) Pub. Date: Jan. 5, 2023

(54) METHOD OF CORRECTING AMPLIFICATION BIAS IN AMPLICON SEQUENCING

(71) Applicant: CELULA CHINA MED-TECHNOLOGY CO., LTD., Chengdu (CN)

(72) Inventors: Di Wu, Chengdu (CN); Haichuan Zhang, San Diego, CA (US)

(73) Assignee: CELULA CHINA MED-TECHNOLOGY CO., LTD., Chengdu, SC (CN)

(21) Appl. No.: 17/943,154

(22) Filed: Sep. 12, 2022

Related U.S. Application Data
(63) Continuation of application No. 16/496,414, filed on Sep. 20, 2019, now abandoned.

(51) Int. Cl. G16B 20/10; G16B 30/00

(52) U.S. Cl. CPC G16B 20/10; G16B 30/00

(57) ABSTRACT

A method to correct amplification bias in amplicon sequencing is disclosed. Amplification efficiency is not constant among different loci in a sample, nor for the same locus in different samples. Differences in 3′-end stability, primer Tm, amplicon length, amplicon GC content, and GC content of amplicon flanking regions all may contribute to amplification bias. Such bias interferes with accurate calculation of copy number for a genomic region of interest and hinders the application of amplicon sequencing for detection of minor copy number variation. The methods of the invention allow correction of amplification bias and enable detection of minor copy number variation using amplicon sequence data.

### FIELD OF THE INVENTION

The present invention relates to computational methods for correcting amplification bias in amplicon sequencing.

### BACKGROUND OF THE INVENTION

Next generation sequencing or massively parallel sequencing typically uses a library generated by multiplex-polymerase chain reaction (PCR). Differences in 3′-end stability, primer melting temperature (Tm), amplicon length, amplicon GC content, and GC content of amplicon flanking regions all may contribute to amplification bias. Such bias interferes with accurate calculation of copy number for a genomic region of interest and hinders the application of amplicon sequencing for detection of minor copy number variation.

Bias can be minimized through careful optimization of factors such as primer design, annealing temperature, buffer composition, and PCR cycle number. See, for example, Markoulatos et al. (2002) J. Clin. Lab. Anal. 16:47-51. Alternatively, raw data can be corrected by computational methods that eliminate amplification bias. However, there remains a need for better methods of correcting bias inherent to multiplex amplification for amplicon sequencing.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The invention is based on the discovery of a novel method for correcting amplification bias. A computational approach is used to eliminate amplification bias in multiplex PCR caused by various factors, including differences in 3′-end stability, primer melting temperature (Tm), amplicon length, amplicon GC content, and GC content of amplicon flanking regions.

In one aspect, the invention includes a method for correcting amplification bias, the method comprising: a) amplifying target nucleic acids; b) acquiring amplicon coverage data for the target nucleic acids; c) calculating a ratio of amplicon coverage between a test genomic region and a reference genomic region for each target nucleic acid; d) removing outliers; e) normalizing the ratio of amplicon coverage between the test genomic region and the reference genomic region for each target nucleic acid according to the formula:

$$normalized ratio = \frac{original ratio}{median ( original ratio )} ;$$

f) calculating differences between the test genomic region and the reference genomic region for primer 3′-end stability (Diff _{3′-end stability}), primer melting temperature (Diff_{Tm}), amplicon length (Diff_{amplicon length}), amplicon GC content (Diff_{Amplicon GC}), and GC content of amplicon flanking sequences (Diff_{Amplicon flank GC}); g) fitting data to obtain regression parameter values A₁, A₂, A₃, A₄ and A₅ according to the formula:

log(normalized ratio of amplicon coverage)=A₁'3Diff_{3′-stability}+A₂×Diff_{Tm}+A₃×Diff_{amplicon length}+A₄×Diff_{Amplicon GC}+A₅×Diff_{Amplicon flank GC}; and h) correcting amplification bias by using the regression parameter values A₁, A₂, A₃, A₄ and A₅ to calculate a predicted logarithmic normalized ratio of amplicon coverage.

In certain embodiments, the target nucleic acids are genomic DNA or RNA. The target nucleic acids may be from a fetus, a child, or an adult. In one embodiment, the target nucleic acids are human. Target nucleic acids may be from a cell, including any type of eukaryotic cell, a prokaryotic cell, or an archaeon cell, a population of cells, a tissue, a virus, an artificial cell, or a cell-free system.

Amplification of target nucleic acids may be performed by any suitable nucleic amplification technique. In one embodiment, amplification comprises performing multiplex polymerase chain reaction (PCR). In another embodiment, amplification comprises performing multiplex reverse transcriptase polymerase chain reaction (RT-PCR).

In certain embodiments, the target nucleic acids are provided in a plurality of samples. In order to facilitate analysis of amplification bias, the amplicon coverage data may be ordered in a matrix as shown in FIG. 1, wherein each row corresponds to a separate amplicon and each column corresponds to a separate sample. A ratio matrix of amplicon coverage may be created from such a data matrix as shown in FIG. 2. Next, the ratio matrix of amplicon coverage may be converted to a normalized ratio matrix of amplicon coverage with row median as shown in FIG. 3.

In another embodiment, the method further comprises detecting copy number variation of at least one target nucleic acid after correcting amplification bias.

In another embodiment, the method further comprises detecting chromosomal aneuploidy after correcting amplification bias.

In another aspect, the invention includes a computer implemented method for correcting amplification bias, the computer performing steps comprising: a) receiving inputted amplicon coverage data for a plurality of target nucleic acids; b) calculating a ratio of amplicon coverage between a test genomic region and a reference genomic region for each target nucleic acid; c) removing outliers; d) normalizing the ratio of amplicon coverage between the test genomic region and the reference genomic region for each target nucleic acid according to the formula:

$$normalized ratio = \frac{original ratio}{median ( original ratio )} ;$$

e) calculating differences between the test genomic region and the reference genomic region for primer 3′-end stability (Diff _{3′-end stability)}, primer melting temperature (Diff_{Tm}), amplicon length (Diff_{amplicon length}), amplicon GC content (Diff_{Amplicon GC}), and GC content of amplicon flanking sequences (Diff_{Amplicon flank GC}); f) fitting data to obtain regression parameter values A₁, A₂, A₃, A₄ and A₅ according to the formula:

log(normalized ratio of amplicon coverage)=A₁×Diff_{3′-end stability}+A₂×Diff_{Tm}+A₃×Diff_{amplicon length}+A₄×Diff_{Amplicon GC}+A₅×Diff_{Amplicon flank GC}; g) correcting amplification bias by using the regression parameter values A₁, A₂, A₃, A₄ and A₅ to calculate a predicted logarithmic normalized ratio of amplicon coverage; and h) displaying information regarding the predicted amplicon coverage with amplification bias correction.

In another embodiment, the computer implemented method further comprises ordering the amplicon coverage data in a matrix as shown in FIG. 1, wherein each row corresponds to a separate amplicon and each column corresponds to a separate sample.

In another embodiment, the computer implemented method further comprises creating a ratio matrix of amplicon coverage as shown in FIG. 2.

In another embodiment, the computer implemented method further comprises creating a normalized ratio matrix of amplicon coverage with row median as shown in FIG. 3.

In another embodiment, the computer implemented method further comprises detecting copy number variation of at least one target nucleic acid after correcting amplification bias.

In another embodiment, the computer implemented method further comprises detecting chromosomal aneuploidy after correcting amplification bias.

A system for correcting amplification bias comprising: a) a storage component for storing amplicon coverage data, wherein the storage component has instructions for correcting the amplification bias stored therein; b) a computer processor for processing data, wherein the computer processor is coupled to the storage component and configured to execute the instructions stored in the storage component in order to receive amplicon coverage data and correct the amplification bias as described herein; and c) a display component for displaying information regarding the predicted amplicon coverage with amplification bias correction.

These and other embodiments of the present invention will readily occur to those of ordinary skill in the art in view of the disclosure herein.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1 shows a data matrix with rows corresponding to amplicons (l to n) and columns corresponding to samples (l to m). The top half of the matrix has data for a test genomic region. The bottom half of the matrix has data for a reference genomic region.

FIG. 2 shows a ratio matrix of amplicon coverage between test and reference genomic regions.

FIG. 3 shows a normalized ratio matrix with row median.

FIGS. 4A and 4B show results of PCR bias correction. FIG. 4A shows the logarithmic normalized ratio of amplicon coverage before and after PCR bias correction for differences in amplicon GC content. FIG. 4A (left) shows a plot of the data using Diff_{amplicon GC} as the X-axis and the logarithmic normalized ratio of amplicon coverage as the Y-axis, each data point representing a unique T/R pair. The color of each data point depends on the loci in the test region of the corresponding T/R pair: light gray represents chromosome 13; medium gray represents chromosome 18; and dark gray represents chromosome 21. Adding the regression line (the gray line) demonstrates the correlation between amplicon GC content and normalized loci coverage. FIG. 4 (at right) is similar except for using the residual c as the Y-axis. Diff_{amplicon GC} is not correlated to the residual c, which indicates that the PCR-bias resulting from the difference of amplicon GC content has been suppressed. FIG. 4B shows a boxplot instead to illustrate the effectiveness of PCR-bias correction in a more intuitive way. Each box represents a chromosome, under ideal conditions, the median of a box should be zero. However, because of the existence of PCR-bias, the box representing chromosome 21 goes down before correction, which may lead to wrong identification. After PCR-bias correction, the box representing chromosome 21 goes up, demonstrating that the correction is effective.

FIG. 5 shows a schematic illustrating the experimental process with application of PCR-bias correction. 10 plasma DNA samples were pooled together, then split into 10 aliquots for amplification to obtain 10 individual sequencing results corrected for PCR bias.

### DESCRIPTION OF THE INVENTION

It is to be understood that the invention is not limited to the particular methodologies, protocols, cell lines, assays, and reagents described herein, as these may vary. It is also to be understood that the terminology used herein is intended to describe particular embodiments of the present invention, and is in no way intended to limit the scope of the present invention as set forth in the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of computer science, statistics, chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18^{th} ed., Mack Publishing Co.; Colowick, S. et al., eds., Methods In Enzymology, Academic Press, Inc.; Handbook of Experimental Immunology, Vols. I-IV (D. M. Weir and C.C. Blackwell, eds., 1986, Blackwell Scientific Publications); Maniatis, T. et al., eds. (1989) Molecular Cloning: A Laboratory Manual, 2^{11d} edition, Vols. I-III, Cold Spring Harbor Laboratory Press; Ausubel, F. M. et al., eds. (1999) Short Protocols in Molecular Biology, 4^{th} edition, John Wiley & Sons; Ream et al., eds. (1998) Molecular Biology Techniques: An Intensive Laboratory Course, Academic Press); M. R. Green and J. Sambrook, et al. (2012) Molecular Cloning: A Laboratory Manual, 4^{th} edition, Cold Spring Harbor Laboratory Press; Newton & Graham, eds. (1997) PCR (Introduction to Biotechniques Series), 2^{11d} edition, Springer Verlag; J. Xu, ed. (2014) Next-generation Sequencing: Current Technologies and Applications, Caister Academic Press; Y. M. Kwon and S. C. Ricke, eds. (2011) High-Throughput Next Generation Sequencing: Methods and Applications (Methods in Molecular Biology), Humana Press; L.C. Wong, ed. (2013) Next Generation Sequencing: Translation to Clinical Diagnostics, Springer.

The present invention relates to the development of a method to correct amplification bias. Amplification efficiency is not constant among different loci in a sample, nor for the same locus in different samples. Differences in 3′-end stability, primer Tm, amplicon length, amplicon GC content, and GC content of amplicon flanking regions all may contribute to amplification bias. Such bias interferes with accurate calculation of copy number for a genomic region of interest and hinders the application of amplicon sequencing for detection of minor copy number variation. The methods of the invention allow correction of amplification bias and enable detection of minor copy number variation using amplicon sequencing data (see Examples).

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description. The invention is capable of other embodiments and of being practiced or of being carried out in various ways. Also, the phraseology and terminology used herein is for the purpose of description and should not be regarded as limiting.

The use of “including,” “comprising,” or “having,” “containing,” “involving,” and variations thereof herein, is meant to encompass the items listed thereafter and equivalents thereof as well as additional items.

It must be noted that as used herein and in the appended claims, the singular forms “a,” “an,” and “the” include plural references unless context clearly dictates otherwise. Thus, for example, a reference to “a nucleic acid” includes a plurality of such nucleic acids, and to equivalents thereof known to those skilled in the art, and so forth.

The term “about,” particularly in reference to a given quantity, is meant to encompass deviations of plus or minus five percent.

As used herein, a “cell” refers to any type of cell isolated from a prokaryotic, eukaryotic, or archaeon organism, including bacteria, archaea, fungi, protists, plants, and animals, including cells from tissues, organs, and biopsies, as well as recombinant cells, cells from cell lines cultured in vitro, and cellular fragments, cell components, or organelles comprising nucleic acids. The term also encompasses artificial cells, such as nanoparticles, liposomes, polymersomes, or microcapsules encapsulating nucleic acids. A cell may include a fixed cell or a live cell.

The terms “nucleic acid,” “nucleic acid molecule,” “polynucleotide,” and “oligonucleotide” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. There is no intended distinction in length between the terms “nucleic acid,” “nucleic acid molecule,” “polynucleotide,” and “oligonucleotide” and these terms will be used interchangeably.

As used herein, the term “target nucleic acid region” or “target nucleic acid” denotes a nucleic acid molecule with a “target sequence” to be amplified. The target nucleic acid may be either single-stranded or double-stranded and may include other sequences besides the target sequence, which may not be amplified. The term “target sequence” refers to the particular nucleotide sequence of the target nucleic acid which is to be amplified. The target sequence may include a probe-hybridizing region contained within the target molecule with which a probe will form a stable hybrid under desired conditions. The “target sequence” may also include the complexing sequences to which the oligonucleotide primers complex and are extended using the target sequence as a template. Where the target nucleic acid is originally single-stranded, the term “target sequence” also refers to the sequence complementary to the “target sequence” as present in the target nucleic acid. If the “target nucleic acid” is originally double-stranded, the term “target sequence” refers to both the plus (+) and minus (−) strands (or sense and anti-sense strands).

The term “primer” or “oligonucleotide primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer can first be treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis. Typically, nucleic acids are amplified using at least one set of oligonucleotide primers comprising at least one forward primer and at least one reverse primer capable of hybridizing to regions of a nucleic acid flanking the portion of the nucleic acid to be amplified.

The term “amplicon” refers to the amplified nucleic acid product of a PCR reaction or other nucleic acid amplification process (e.g., ligase chain reaction (LGR), nucleic acid sequence based amplification (NASBA), transcription-mediated amplification (TMA), Q-beta amplification, strand displacement amplification, or target mediated amplification). DNA amplicons may be generated from RNA by RT-PCR.

As used herein, the term “probe” or “oligonucleotide probe” refers to a polynucleotide, as defined above, that contains a nucleic acid sequence complementary to a nucleic acid sequence present in the target nucleic acid analyte. The polynucleotide regions of probes may be composed of DNA, and/or RNA, and/or synthetic nucleotide analogs. Probes may be labeled in order to detect the target sequence. Such a label may be present at the 5′ end, at the 3′ end, at both the 5′ and 3′ ends, and/or internally. The “oligonucleotide probe” may contain at least one fluorescer and at least one quencher. Quenching of fluorophore fluorescence may be eliminated by exonuclease cleavage of the fluorophore from the oligonucleotide (e.g., TaqMan assay) or by hybridization of the oligonucleotide probe to the nucleic acid target sequence (e.g., molecular beacons). Additionally, the oligonucleotide probe will typically be derived from a sequence that lies between the sense and the antisense primers when used for nucleic acid amplification.

It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under conditions, generally where there is about 90% or greater homology.

The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis.

The “melting temperature” or “Tₘ” of double-stranded DNA is defined as the temperature at which half of the helical structure of the DNA is lost due to heating or other dissociation of the hydrogen bonding between base pairs, for example, by acid or alkali treatment, or the like. The Tₘ of a DNA molecule depends on its length and on its base composition. DNA molecules rich in GC base pairs have a higher Tₘ than those having an abundance of AT base pairs. Separated complementary strands of DNA spontaneously reassociate or anneal to form duplex DNA when the temperature is lowered below the Tₘ. The highest rate of nucleic acid hybridization occurs approximately 25 degrees C. below the Tₘ. The T. may be estimated using the following relationship: Tₘ=69.3+0.41(GC) % (Marmur et al. (1962) J Mol. Biol. 5:109-118).

As used herein, a “biological sample” refers to a sample of cells, tissue, or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, cells, muscles, joints, organs, biopsies and also samples of in vitro cell culture constituents including but not limited to conditioned media resulting from the growth of cells and tissues in culture medium, e.g., recombinant cells, artificial cells, and cell components.

The term “subject” includes any invertebrate or vertebrate subject, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like, insects, nematodes, fish, amphibians, and reptiles. The term does not denote a particular age. Thus, both adult and newborn individuals are intended to be covered.

### Correction of Amplification Bias

The methods of the invention may be used to correct bias in sequencing libraries generated by multiplex amplification of nucleic acids. The method typically comprises first acquiring amplicon coverage data for target nucleic acids of interest. Next, the ratio of amplicon coverage between a test genomic region and a reference genomic region for each target nucleic acid is calculated. Outliers are removed followed by data normalization. The ratio of amplicon coverage between the test genomic region and the reference genomic region for each target nucleic acid is normalized according to the formula:

$$normalized ratio = \frac{original ratio}{median ( original ratio )} .$$

In order to correct amplification bias, various parameters that may contribute to amplification bias are evaluated by analyzing sequence differences between the test and reference genomic regions. Differences in primer 3′-end stability (Diff_{3′-end stability}), primer melting temperature (Diff_{Tm}), amplicon length (Diff_{amplicon length}), amplicon GC content (Diff_{Amplicon GC}), and GC content of amplicon flanking sequences (Diff_{Amplicon flank GC}) are calculated. Regression parameter values A₁, A₂, A₃, A₄ and A₅ are obtained by fitting the data according to the formula: log(normalized ratio o f amplicon coverage)=A₁×Diff_{3′-end stability}+A₂×Diff_{Tm}+A₃×Diff_{amplicon length}+A₄×Diff_{Amplicon GC}+A₅×Diff_{Amplicon flank GC}. The regression parameter values A₁, A₂, A₃, A₄ and A₅ are used to calculate a predicted logarithmic normalized ratio of amplicon coverage that is corrected for amplification bias.

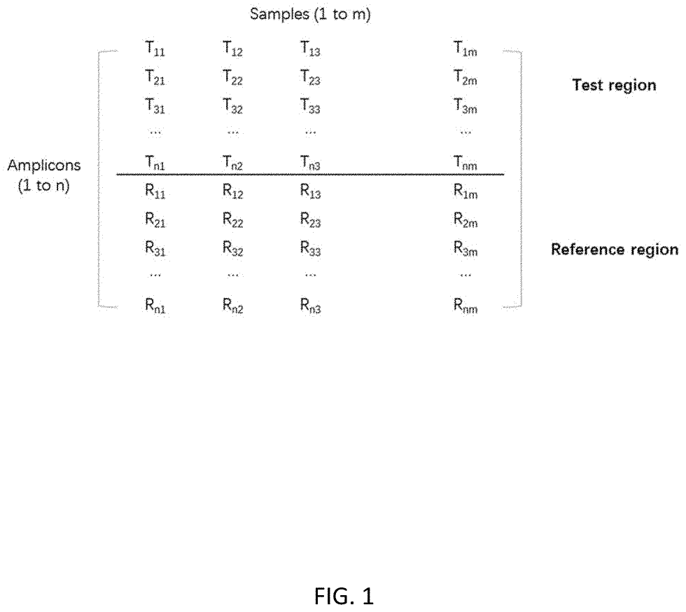

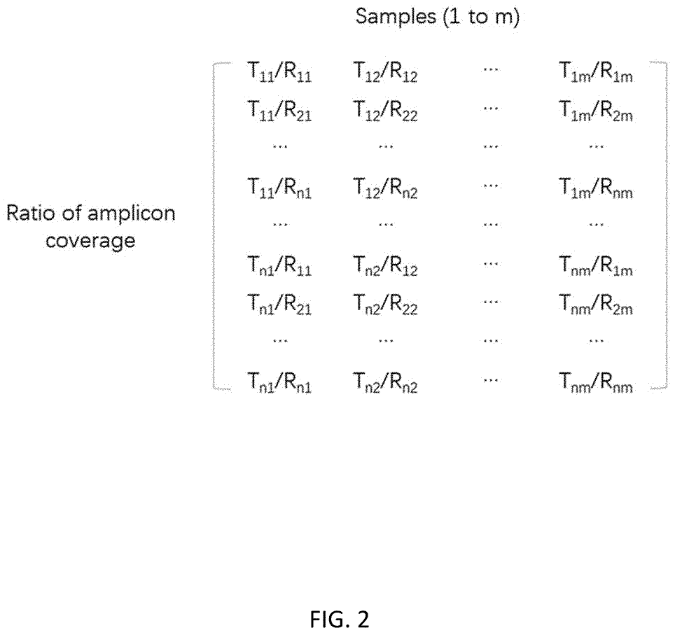

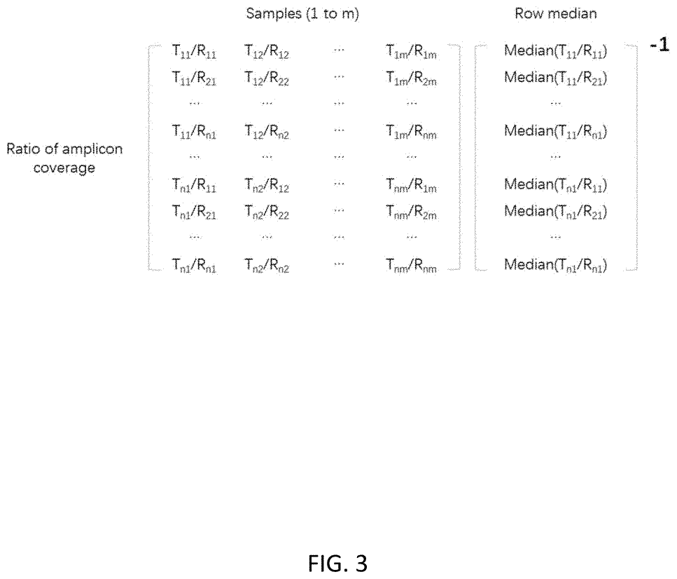

In certain embodiments, the target nucleic acids to be amplified are provided in a plurality of samples. In order to facilitate analysis of amplification bias, the amplicon coverage data may be ordered in a matrix as shown in FIG. 1, wherein each row corresponds to a separate amplicon and each column corresponds to a separate sample. A ratio matrix of amplicon coverage may be created from such a data matrix as shown in FIG. 2. Next, the ratio matrix of amplicon coverage may be converted to a normalized ratio matrix of amplicon coverage with row median as shown in FIG. 3.

Nucleic acids to be amplified and sequenced may be genomic DNA or cDNA (i.e., derived from RNA by reverse transcription). Sources of nucleic acid molecules include, but are not limited to, organelles, cells, tissues, organs, and organisms. For example, a biological sample containing nucleic acids to be analyzed can be any sample of cells, tissue, or fluid isolated from a prokaryotic, archaeon, or eukaryotic organism, including but not limited to, for example, blood, saliva, cells from buccal swabbing, fecal matter, urine, bone marrow, bile, spinal fluid, lymph fluid, sputum, ascites, bronchial lavage fluid, synovial fluid, samples of the skin, external secretions of the skin, respiratory, intestinal, and genitourinary tracts, tears, saliva, milk, organs, biopsies, and also samples of cells, including cells from bacteria, archaea, fungi, protists, plants, and animals as well as in vitro cell culture constituents, including recombinant cells and tissues grown in culture medium. A biological sample may also contain nucleic acids from viruses. In certain embodiments, nucleic acids (e.g., DNA or RNA) are obtained from a single cell or a selected population of cells of interest. The cell may be a live cell or a fixed cell. In certain embodiments, the cell is an invertebrate cell, vertebrate cell, yeast cell, mammalian cell, rodent cell, primate cell, or human cell. Additionally, the cell may be a genetically aberrant cell, rare blood cell, or cancerous cell. The target nucleic acids may be from a fetus, a child, or an adult.

Cells may be pre-treated in any number of ways prior to amplification and sequencing of nucleic acids (e.g., DNA and/or RNA). For instance, in certain embodiments, the cell may be treated to disrupt (or lyse) the cell membrane, for example, by treating samples with one or more detergents (e.g., Triton-X-100, Tween 20, Igepal CA-630, NP-40, Brij 35, and sodium dodecyl sulfate) and/or denaturing agents (e.g., guanidinium agents). In cell types with cell walls, such as yeast and plants, initial removal of the cell wall may be necessary to facilitate cell lysis. Cell walls can be removed, for example, using enzymes, such as cellulases, chitinases, or bacteriolytic enzymes, such as lysozyme (destroys peptidoglycans), mannase, and glycanase. As will be clear to one of skill in the art, the selection of a particular enzyme for cell wall removal will depend on the cell type under study.

After lysing, nucleic acid extraction from cells may be performed using conventional techniques, such as phenol-chloroform extraction, precipitation with alcohol, or non-specific binding to a solid phase (e.g., silica). Care should be taken to avoid shearing the nucleic acids to be sequenced during extraction steps. Additionally, enzymatic or chemical methods may be used to remove contaminating cellular components (e.g., ribosomal RNA, mitochondrial RNA, protein, or other macromolecules). For example, proteases can be used to remove contaminating proteins. A nuclease inhibitor may be used to prevent degradation of nucleic acids.

DNA may be amplified prior to sequencing using any suitable polymerase chain reaction (PCR) technique known in the art. In PCR, a pair of primers is employed in excess to hybridize to the complementary strands of a target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well known in the art and has been described in, e.g., Innis et al. (eds.) PCR Protocols (Academic Press, NY 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818, all incorporated herein by reference in their entireties.

In particular, PCR uses relatively short oligonucleotide primers which flank the target nucleotide sequence to be amplified, oriented such that their 3′ ends face each other, each primer extending toward the other. Typically, the primer oligonucleotides are in the range of between 10-100 nucleotides in length, such as 15-60, 20-40 and so on, more typically in the range of between 20-40 nucleotides long, and any length between the stated ranges.

The DNA is extracted and denatured, preferably by heat, and hybridized with first and second primers that are present in molar excess. Polymerization is catalyzed in the presence of the four deoxyribonucleotide triphosphates (dNTPs—dATP, dGTP, dCTP and dTTP) using a primer- and template-dependent polynucleotide polymerizing agent, such as any enzyme capable of producing primer extension products, for example, E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). This results in two “long products” which contain the respective primers at their 5′ ends covalently linked to the newly synthesized complements of the original strands. The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, an additional two long products are produced, and a number of short products equal to the number of long and short products remaining at the end of the previous cycle. Thus, the number of short products containing the target sequence grows exponentially with each cycle. Preferably, PCR is carried out with a commercially available thermal cycler (available from, e.g., Bio-Rad, Applied Biosystems, and Qiagen).

RNA may be amplified by reverse transcribing RNA into cDNA with a reverse transcriptase and then performing PCR (i.e., RT-PCR), as described above. Suitable reverse transcriptases include avian myeloblastosis virus (AMV) reverse transcriptase and Moloney murine leukemia virus (MMLV) reverse transcriptase (available from, e.g., Promega, New England Biolabs, and Thermo Fisher Scientific Inc.). Alternatively, a single enzyme may be used for both steps as described in U.S. Pat. No. 5,322,770, incorporated herein by reference in its entirety. In this manner, cDNA can be generated from all types of RNA, including mRNA, non-coding RNA, microRNA, siRNA, and viral RNA to allow sequencing of RNA transcripts.

In certain embodiments, amplification comprises performing a clonal amplification method, such as, but not limited to bridge amplification, emulsion PCR (ePCR), or rolling circle amplification. In particular, clonal amplification methods such as, but not limited to bridge amplification, emulsion PCR (ePCR), or rolling circle amplification may be used to cluster amplified nucleic acids in a discrete area (see, e.g., U.S. Pat. Nos. 7,790,418; 5,641,658; 7,264,934; 7,323,305; 8,293,502; 6,287,824; and International Application WO 1998/044151 A1; Lizardi et al. (1998) Nature Genetics 19: 225-232; Leamon et al. (2003) Electrophoresis 24: 3769-3777; Dressman et al. (2003) Proc. Natl. Acad. Sci. USA 100: 8817-8822; Tawfik et al. (1998) Nature Biotechnol. 16: 652-656; Nakano et al. (2003) J. Biotechnol. 102: 117-124; herein incorporated by reference). For this purpose, adapter sequences (e.g., adapters with sequences complementary to universal amplification primers or bridge PCR amplification primers) suitable for high-throughput amplification may be added to DNA or cDNA fragments at the 5′ and 3′ends. For example, bridge PCR primers, attached to a solid support, can be used to capture DNA templates comprising adapter sequences complementary to the bridge PCR primers. The DNA templates can then be amplified, wherein the amplified products of each DNA template cluster in a discrete area on the solid support.

In particular, the methods of the invention are applicable to digital PCR methods. For digital PCR, a sample containing nucleic acids is separated into a large number of partitions before performing PCR. Partitioning can be achieved in a variety of ways known in the art, for example, by use of micro well plates, capillaries, emulsions, arrays of miniaturized chambers or nucleic acid binding surfaces. Separation of the sample may involve distributing any suitable portion including up to the entire sample among the partitions. Each partition includes a fluid volume that is isolated from the fluid volumes of other partitions. The partitions may be isolated from one another by a fluid phase, such as a continuous phase of an emulsion, by a solid phase, such as at least one wall of a container, or a combination thereof. In certain embodiments, the partitions may comprise droplets disposed in a continuous phase, such that the droplets and the continuous phase collectively form an emulsion.

The partitions may be formed by any suitable procedure, in any suitable manner, and with any suitable properties. For example, the partitions may be formed with a fluid dispenser, such as a pipette, with a droplet generator, by agitation of the sample (e.g., shaking, stirring, sonication, etc.), and the like. Accordingly, the partitions may be formed serially, in parallel, or in batch. The partitions may have any suitable volume or volumes. The partitions may be of substantially uniform volume or may have different volumes. Exemplary partitions having substantially the same volume are monodisperse droplets. Exemplary volumes for the partitions include an average volume of less than about 100, 10 or 1 μL, less than about 100, 10, or 1 nL, or less than about 100, 10, or 1 pL, among others.

After separation of the sample, PCR is carried out in the partitions. The partitions, when formed, may be competent for performance of one or more reactions in the partitions. Alternatively, one or more reagents may be added to the partitions after they are formed to render them competent for reaction. The reagents may be added by any suitable mechanism, such as a fluid dispenser, fusion of droplets, or the like.

After PCR amplification, nucleic acids are quantified by counting the partitions that contain PCR amplicons. Partitioning of the sample allows quantification of the number of different molecules by assuming that the population of molecules follows a Poisson distribution. For a description of digital PCR methods, see, e.g., Hindson et al. (2011) Anal. Chem. 83(22):8604-8610; Pohl and Shih (2004) Expert Rev. Mol. Diagn. 4(1):41-47; Pekin et al. (2011) Lab Chip 11 (13): 2156-2166; Pinheiro et al. (2012) Anal. Chem. 84 (2): 1003-1011; Day et al. (2013) Methods 59(1):101-107; herein incorporated by reference in their entireties.

Oligonucleotides, including primers and probes can be readily synthesized by standard techniques, e.g., solid phase synthesis via phosphoramidite chemistry, as disclosed in U.S. Pat. Nos. 4,458,066 and 4,415,732, incorporated herein by reference; Beaucage et al. Tetrahedron (1992) 48:2223-2311; and Applied Biosystems User Bulletin No. 13 (1 Apr. 1987). Other chemical synthesis methods include, for example, the phosphotriester method described by Narang et al. Meth. Enzymol. (1979) 68:90 and the phosphodiester method disclosed by Brown et al. Meth. Enzymol. (1979) 68:109. Poly(A) or poly(C), or other non-complementary nucleotide extensions may be incorporated into oligonucleotides using these same methods. Hexaethylene oxide extensions may be coupled to the oligonucleotides by methods known in the art. Cload et al. J. Am. Chem. Soc. (1991) 113:6324-6326; U.S. Pat. No. 4,914,210 to Levenson et al.; Durand et al. Nucleic Acids Res. (1990) 18:6353-6359; and Horn et al. Tet. Lett. (1986) 27:4705-4708.

Moreover, the oligonucleotides (e.g., primers and probes) may be coupled to labels for detection. There are several means known for derivatizing oligonucleotides with reactive functionalities which permit the addition of a label. For example, several approaches are available for biotinylating probes so that radioactive, fluorescent, chemiluminescent, enzymatic, or electron dense labels can be attached via avidin. See, e.g., Broken et al. Nucl. Acids Res. (1978) 5:363-384 which discloses the use of ferritin-avidin-biotin labels; and Chollet et al. Nucl. Acids Res. (1985) 13:1529-1541 which discloses biotinylation of the 5′ termini of oligonucleotides via an aminoalkylphosphoramide linker arm. Several methods are also available for synthesizing amino-derivatized oligonucleotides which are readily labeled by fluorescent or other types of compounds derivatized by amino-reactive groups, such as isothiocyanate, N-hydroxysuccinimide, or the like, see, e.g., Connolly, Nucl. Acids Res. (1987) 15:3131-3139, Gibson et al. Nucl. Acids Res. (1987) 15:6455-6467 and U.S. Pat. No. 4,605,735 to Miyoshi et al. Methods are also available for synthesizing sulfhydryl-derivatized oligonucleotides, which can be reacted with thiol-specific labels, see, e.g., U.S. Pat. No. 4,757,141 to Fung et al., Connolly et al. Nucl. Acids Res. (1985) 13:4485-4502 and Spoat et al. Nucl. Acids Res. (1987) 15:4837-4848. A comprehensive review of methodologies for labeling DNA fragments is provided in Matthews et al. Anal. Biochem. (1988) 169:1-25.

For example, oligonucleotides may be fluorescently labeled by linking a fluorescent molecule to the non-ligating terminus of the molecule. Guidance for selecting appropriate fluorescent labels can be found in Smith et al. Meth. Enzymol. (1987) 155:260-301; Karger et al. Nucl. Acids Res. (1991) 19:4955-4962; Guo et al. (2012) Anal. Bioanal. Chem. 402(10):3115-3125; and Molecular Probes Handbook, A Guide to Fluorescent Probes and Labeling Technologies, 11^{th} edition, Johnson and Spence eds., 2010 (Molecular Probes/Life Technologies). Fluorescent labels include fluorescein and derivatives thereof, such as disclosed in U.S. Pat. No. 4,318,846 and Lee et al. Cytometry (1989) 10:151-164. Dyes for use in the present invention include 3-phenyl-7-isocyanatocoumarin, acridines, such as 9-isothiocyanatoacridine and acridine orange, pyrenes, benzoxadiazoles, and stilbenes, such as disclosed in U.S. Pat. No. 4,174,384. Additional dyes include SYBR green, SYBR gold, Yakima Yellow, Texas Red, 3-(c-carboxypentyl)-3′-ethyl-5,5′-dimethyloxa-carbocyanine (CYA); 6-carboxy fluorescein (FAM); CAL Fluor Orange 560, CAL Fluor Red 610, Quasar Blue 670; 5,6-carboxyrhodamine-110 (R110); 6-carboxyrhodamine-6G (R6G); N′,N′,N′,N′-tetramethyl-6-carboxyrhodamine (TAMRA); 6-carboxy-X-rhodamine (ROX); 2′, 4′, 5′, 7′, -tetrachloro-4-7-dichlorofluorescein (TET); 2′, 7′-dimethoxy-4′, 5′-6 carboxyrhodamine (JOE); 6-carboxy-2′,4,4′,5′,7,7′-hexachlorofluorescein (HEX); Dragonfly orange; ATTO-Tec; Bodipy; ALEXA; VIC, Cy3, and Cy5. These dyes are commercially available from various suppliers such as Life Technologies (Carlsbad, Calif.), Biosearch Technologies (Novato, Calif.), and Integrated DNA Technolgies (Coralville, Iowa). Fluorescent labels include fluorescein and derivatives thereof, such as disclosed in U.S. Pat. No. 4,318,846 and Lee et al. Cytometry (1989) 10:151-164, and 6-FAM, JOE, TAMRA, ROX, HEX-1, HEX-2, ZOE, TET-1 or NAN-2, and the like.

Oligonucleotides can also be labeled with a minor groove binding (MGB) molecule, such as disclosed in U.S. Pat. Nos. 6,884,584, 5,801,155; Afonina et al. (2002) Biotechniques 32:940-944, 946-949; Lopez-Andreo et al. (2005) Anal. Biochem. 339:73-82; and Belousov et al. (2004) Hum Genomics 1:209-217. Oligonucleotides having a covalently attached MGB are more sequence specific for their complementary targets than unmodified oligonucleotides. In addition, an MGB group increases hybrid stability with complementary DNA target strands compared to unmodified oligonucleotides, allowing hybridization with shorter oligonucleotides.

Additionally, oligonucleotides can be labeled with an acridinium ester (AE) using the techniques described below. Current technologies allow the AE label to be placed at any location within the probe. See, e.g., Nelson et al. (1995) “Detection of Acridinium Esters by Chemiluminescence” in Nonisotopic Probing, Blotting and Sequencing, Kricka L. J. (ed.) Academic Press, San Diego, Calif.; Nelson et al. (1994) “Application of the Hybridization Protection Assay (HPA) to PCR” in The Polymerase Chain Reaction, Mullis et al. (eds.) Birkhauser, Boston, Mass.; Weeks et al. Clin. Chem. (1983) 29:1474-1479; Berry et al. Clin. Chem. (1988) 34:2087-2090. An AE molecule can be directly attached to the probe using non-nucleotide-based linker arm chemistry that allows placement of the label at any location within the probe. See, e.g., U.S. Pat. Nos. 5,585,481 and 5,185,439.

DNA or cDNA molecules may be further purified by immobilization on a solid support, such as silica, adsorbent beads (e.g., oligo(dT) coated beads or beads composed of polystyrene-latex, glass fibers, cellulose or silica), magnetic beads, or by reverse phase, gel filtration, ion-exchange, or affinity chromatography. Alternatively, an electric field-based method can be used to separate DNA/cDNA fragments from other molecules. Exemplary electric field-based methods include polyacrylamide gel electrophoresis, agarose gel electrophoresis, capillary electrophoresis, and pulsed field electrophoresis. See, e.g., U.S. Pat. Nos. 5,234,809; 6,849,431; 6,838,243; 6,815,541; and 6,720,166; and Sambrook et al. Molecular Cloning: A Laboratory Manual (3^{rd} Edition, 2001); Recombinant DNA Methodology (Selected Methods in Enzymology, R. Wu, L. Grossman, K. Moldave eds., Academic Press, 1989); J. Kieleczawa DNA Sequencing II: Optimizing Preparation And Cleanup (Jones & Bartlett Learning; 2^{nd} edition, 2006); herein incorporated by reference in their entireties.

### Sequencing

Any high-throughput technique for sequencing the nucleic acids can be used in the practice of the invention. DNA sequencing techniques include dideoxy sequencing reactions (Sanger method) using labeled terminators or primers and gel separation in slab or capillary, sequencing by synthesis using reversibly terminated labeled nucleotides, pyrosequencing, 454 sequencing, sequencing by synthesis using allele specific hybridization to a library of labeled clones followed by ligation, real time monitoring of the incorporation of labeled nucleotides during a polymerization step, polony sequencing, SOLID sequencing, and the like.

Certain high-throughput methods of sequencing comprise a step in which individual molecules are spatially isolated on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)). Such methods may comprise amplifying the isolated molecules either before or after they are spatially isolated on a solid surface. Prior amplification may comprise emulsion-based amplification, such as emulsion PCR, or rolling circle amplification.

Of particular interest is sequencing on the Illumina MiSeq, NextSeq, and HiSeq platforms, which use reversible-terminator sequencing by synthesis technology (see, e.g., Shen et al. (2012) BMC Bioinformatics 13:160; Junemann et al. (2013) Nat. Biotechnol. 31(4):294-296; Glenn (2011) Mol. Ecol. Resour. 11(5):759-769; Thudi et al. (2012) Brief Funct. Genomics 11(1):3-11; herein incorporated by reference).

### Applications

The methods of the invention will be especially useful in genetic screening for aneuploidy and/or copy number variation associated with various diseases, structural abnormalities, and/or genetic lethality. Correction of amplification bias in sequencing data, as described herein, makes possible more accurate detection of even minor copy number variation. In particular, the methods will find use in non-invasive prenatal testing to detect fetal chromosomal aneuploidy or copy number variation. A biological sample can be collected from the mother or potential mother of an offspring prior to conception or after conception and analyzed. Detection of aneuploidy or copy number variation, as described herein, may indicate an increased risk of the offspring developing abnormally or having a disease (e.g., Down Syndrome (Trisomy 21), Edwards Syndrome (Trisomy 18), or Patau Syndrome (Trisomy 13)). The offspring may be, for example, a neonate or a fetus. In particular, this method can be used to evaluate a mother or potential mother potentially at high risk of having a child with a disease associated with aneuploidy or copy number variation, such as a mother or potential mother who has had a previous child with such a disease or a familial history of the disease, or a history of miscarriages.

The methods of the invention will also find use in genetic testing of cancerous cells. Aneuploidy and copy number variation are commonly associated with many types of cancer. Hence, genetic testing of cancerous cells or abnormal potentially precancerous cells may be useful for diagnosing a patient with a particular type of cancer or precancerous condition and determining an appropriate treatment regimen.

For genetic testing, a biological sample containing nucleic acids is collected from an individual. The biological sample is typically blood, saliva, or cells from buccal swabbing or a biopsy, but can be any sample from bodily fluids, tissue, or cells that contains genomic DNA or RNA of the individual. For prenatal testing of a fetus, the biological sample can be, for example, amniotic fluid (e.g., amniocentesis), placental tissue (e.g., chorionic villus sampling), or fetal blood (e.g., umbilical cord blood sampling). In particular, non-invasive cell-free fetal DNA in maternal blood or nucleic acids extracted from fetal cells in maternal blood (FCMB) can be used in genetic screening. The methods of the invention are also applicable to genetic screening of embryos produced by in vitro fertilization (IVF). For example, preimplantation genetic diagnosis (PGD) can be performed using the methods described herein to correct amplification bias in order to improve detection of aneuploidy and/or copy number variation in embryos prior to transfer to a mother. In certain embodiments, nucleic acids from the biological sample are isolated and/or purified prior to amplification, sequencing, and analysis using methods well-known in the art. See, e.g., Green and Sambrook Molecular Cloning: A Laboratory Manual (Cold Spring Harbor Laboratory Press; 4^{th} edition, 2012); and Current Protocols in Molecular Biology (Ausubel ed., John Wiley & Sons, 1995); herein incorporated by reference in their entireties.

Copy number variation can be evaluated based on “relative copy number” so that apparent differences in gene copy numbers in different samples are not distorted by differences in sample amounts. The relative copy number of a gene (per genome) can be expressed as the ratio of the copy number of a target gene to the copy number of a reference polynucleotide sequence in a DNA sample. The reference polynucleotide sequence can be a sequence having a known genomic copy number. Typically, the reference sequence will have a single genomic copy and is a sequence that is not likely to be amplified or deleted in the genome. It is not necessary to empirically determine the copy number of a reference sequence. Rather, the copy number may be assumed based on the normal copy number in the organism of interest. Accordingly, the relative copy number of the target nucleotide sequence in a DNA sample is calculated from the ratio of the two genes. wherein detection of copy number variation, that is, the presence of a greater or fewer number of a gene (i.e., abnormal copy number) in the subject compared to a control subject (e.g., normal, healthy subject) is diagnostic of a disease.

### System and Computerized Methods for Correcting Amplification Bias

In a further aspect, the invention includes a computer implemented method for correcting amplification bias. The computer performs steps comprising: a) receiving inputted amplicon coverage data for a plurality of target nucleic acids; b) calculating a ratio of amplicon coverage between a test genomic region and a reference genomic region for each target nucleic acid; c) removing outliers; d) normalizing the ratio of amplicon coverage between the test genomic region and the reference genomic region for each target nucleic acid according to the formula:

$$normalized ratio = \frac{original ratio}{median ( original ratio )} ;$$

e) calculating differences between the test genomic region and the reference genomic region for primer 3′-end stability (Diff_{3′-end stability}), primer melting temperature (Diff_{Tm}), amplicon length (Diff_{amplicon length}), amplicon GC content (Diff_{Amplicon GC}), and GC content of amplicon flanking sequences (Diff_{Amplicon flank GC}); f) fitting data to obtain regression parameter values A₁, A₂, A₃, A₄ and A₅ according to the formula:

log(normalized ratio of amplicon coverage)=A₁×Diff_{3′-end stability}+A₂×Diff_{Tm}+A₃×Diff_{amplicon length}+A₄×Diff_{Amplicon GC}+A₅×Diff_{Amplicon flank GC};

g) correcting amplification bias by using the regression parameter values A₁, A₂, A₃, A₄ and A₅ to calculate a predicted logarithmic normalized ratio of amplicon coverage; and h) displaying information regarding the predicted amplicon coverage with amplification bias correction.

In some embodiments, amplicon coverage data is for target nucleic acids from a plurality of samples. In certain embodiments, the computer implemented method further comprises creating a data matrix, as shown in FIG. 1, to organize data from multiple samples, wherein each row of the matrix corresponds to a separate amplicon and each column of the matrix corresponds to a separate sample. A ratio matrix of amplicon coverage is next created from such a data matrix as shown in FIG. 2, and the ratio matrix of amplicon coverage is converted to a normalized ratio matrix of amplicon coverage with the row median as shown in FIG. 3.

In another embodiment, the computer implemented method further comprises detecting chromosomal aneuploidy and/or copy number variation of at least one sequence after correcting for amplification bias.

In a further aspect, the invention includes a system for performing the computer implemented method to correct amplification bias, as described herein. A system for correcting amplification bias may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for correcting the amplification bias, as described herein (see Examples). The computer processor is coupled to the storage component and configured to execute the instructions stored in the storage component in order to receive amplicon coverage data and correct amplification bias as described herein. The display component displays information regarding the predicted amplicon coverage with amplification bias correction.

The storage component may be of any type capable of storing information accessible by the processor, such as a hard-drive, memory card, ROM, RAM, DVD, CD-ROM, Blu-ray, USB Flash drive, write-capable, and read-only memories. The processor may be any well-known processor, such as processors from Intel Corporation. Alternatively, the processor may be a dedicated controller such as an ASIC.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the system for correction of amplification bias is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on a removable DVD, and others within a read-only computer chip. Some or all of the instructions and data may be stored in a location physically remote from, yet still accessible by, the processor. Similarly, the processor may actually comprise a collection of processors which may or may not operate in parallel.

In one aspect, the computer is a server communicating with one or more client computers. Each client computer may be configured similarly to the server, with a processor, storage component and instructions. Each client computer may be a personal computer, intended for use by a person, having all the internal components normally found in a personal computer such as a central processing unit (CPU), display (for example, a monitor displaying information processed by the processor), DVD, hard-drive, user input device (for example, a mouse, keyboard, touch-screen or microphone), speakers, modem and/or network interface device (telephone, cable or otherwise) and all of the components used for connecting these elements to one another and permitting them to communicate (directly or indirectly) with one another. Moreover, computers in accordance with the systems and methods described herein may comprise any device capable of processing instructions and transmitting data to and from humans and other computers including network computers lacking local storage capability.

Although the client computers may comprise a full-sized personal computer, many aspects of the system and method are particularly advantageous when used in connection with mobile devices capable of wirelessly exchanging data with a server over a network such as the Internet. For example, client computer may be a wireless-enabled PDA such as a Blackberry phone, Apple iPhone, Android phone, or other Internet-capable cellular phone. In such regard, the user may input information using a small keyboard, a keypad, a touch screen, or any other means of user input. The computer may have an antenna for receiving a wireless signal.

The server and client computers are capable of direct and indirect communication, such as over a network. It should be appreciated that a typical system can include a large number of connected computers, with each different computer being at a different node of the network. The network, and intervening nodes, may comprise various combinations of devices and communication protocols including the Internet, World Wide Web, intranets, virtual private networks, wide area networks, local networks, cell phone networks, private networks using communication protocols proprietary to one or more companies, Ethernet, WiFi and HTTP. Such communication may be facilitated by any device capable of transmitting data to and from other computers, such as modems (e.g., dial-up or cable), networks and wireless interfaces. The server may be a web server.

Although certain advantages are obtained when information is transmitted or received as noted above, other aspects of the system and method are not limited to any particular manner of transmission of information. For example, in some aspects, information may be sent via a medium such as a disk, tape, flash drive, memory card, DVD, Blu-Ray, or CD-ROM. In other aspects, the information may be transmitted in a non-electronic format and manually entered into the system. Yet further, although some functions are indicated as taking place on a server and others on a client, various aspects of the system and method may be implemented by a single computer having a single processor.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications are intended to fall within the scope of the appended claims.

### Example 1

### Correcting Amplification Bias of Multiplex PCR for Fetal Aneuploidy Detection

Here we describe computational methods for correction of amplification bias and their application to non-invasive prenatal testing using maternal cell-free DNA to aid detection of fetal chromosomal aneuploidy. Amplification bias of an 1855-plex PCR was corrected to allow fetal aneuploidy detection using maternal blood with as little as 4% fetal DNA.

Correction of amplification bias for amplicon sequencing was performed as follows:
1. Amplicon coverage of each tested sample is acquired; then data is entered into a matrix with each row representing each amplicon and each column representing each sample as shown in FIG. 1.
2. A ratio matrix (FIG. 2) is produced from the data matrix generated in step 1 by calculating the ratio of amplicon coverage for every combination between a test genomic region and a reference genomic region. The amplicon coverage of the test region is the numerator and the amplicon coverage of the reference region is the denominator. For example, given amplicons of the test region: T1, T2 and T3 and amplicons of the reference region: R1, R2 and R3, the coverage ratio generated comprises: T1/R1, T1/R2, T1/R3, T2/R1, T2/R2, T2/R3, T3/R1, T3/R2, T3/R3.
3. Outliers are removed by row for the ratio matrix generated in step 2.
4. The result of step 3 is normalized by row using the formula:

Normalized ratio=(Original ratio)/(median(Original ratio))
5. Differences in primer 3′-end stability, primer Tm, amplicon length, amplicon GC content and amplicon flanking 200 bp GC content are calculated for every combination between the test and reference genomic region. Parameters for the amplicons of the test region should be at the left side of the minus, while parameters for the amplicons of the reference region should be on the right side of the minus. For example, given amplicons of the test region: T1, T2 and T3 and amplicons of the reference region: R1, R2 and R3, attribute difference comprises: T1-R1, T1-R2, T1-R3, T2-R1, T2-R2, T2-R3, T3-R1, T3-R2, T3-R3
6. The regression parameters, A₁, A₂, A₃, A₄ and A₅, are obtained by fitting of the following formula with the results of steps 4 and 5:

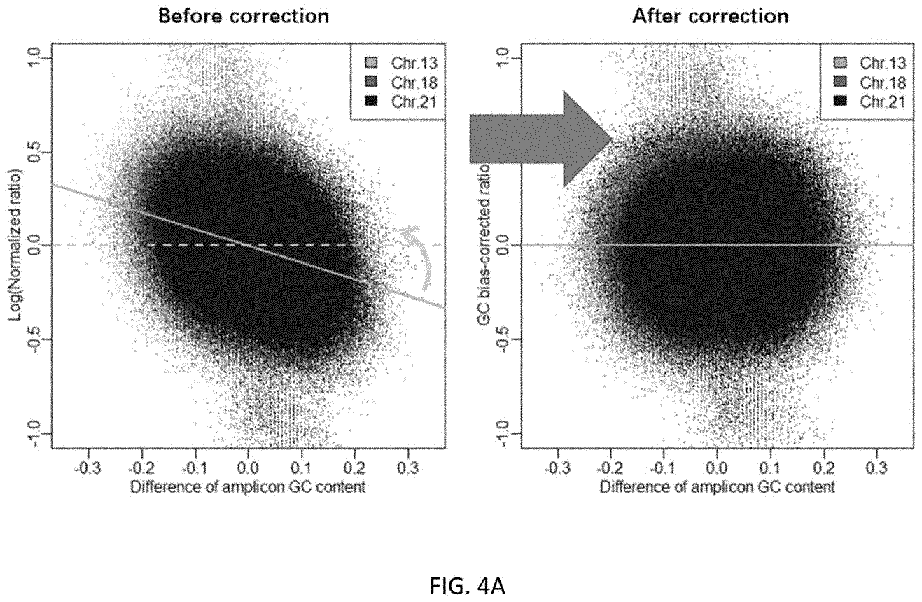

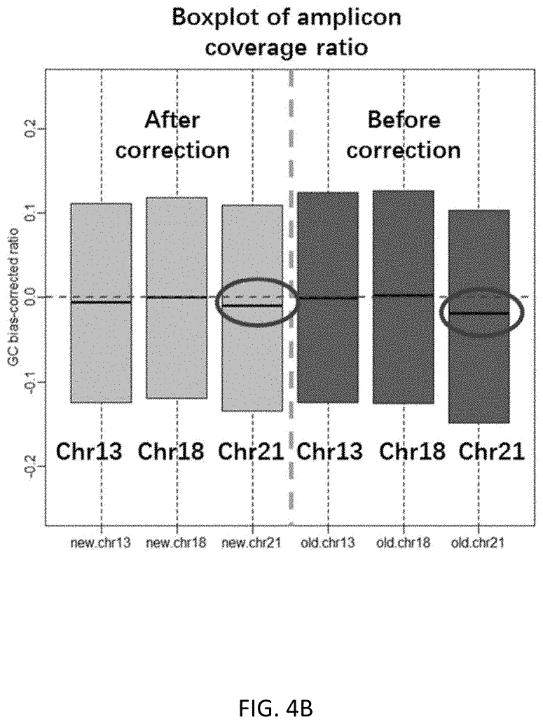

log(Normalized Ratio of Amplicon Coverage)=A₁×Diff_{3′-end stability}A₂×Diff_{Tm}+A₃×Diff_{amplicon length}A₄×Diff_{Amplicon GC}+A₅×Diff_{Amplicon flank GC}
7. The regression parameters acquired by step 6 are used to obtain the predicted logarithmic normalized ratio of amplicon coverage, which then can be used to calculate the difference between an observation value and prediction value, which has been corrected for amplification bias (FIG. 4).

### Example 2

### Correcting Amplification Bias of Multiplex PCR for Pooled Plasma-DNA Samples

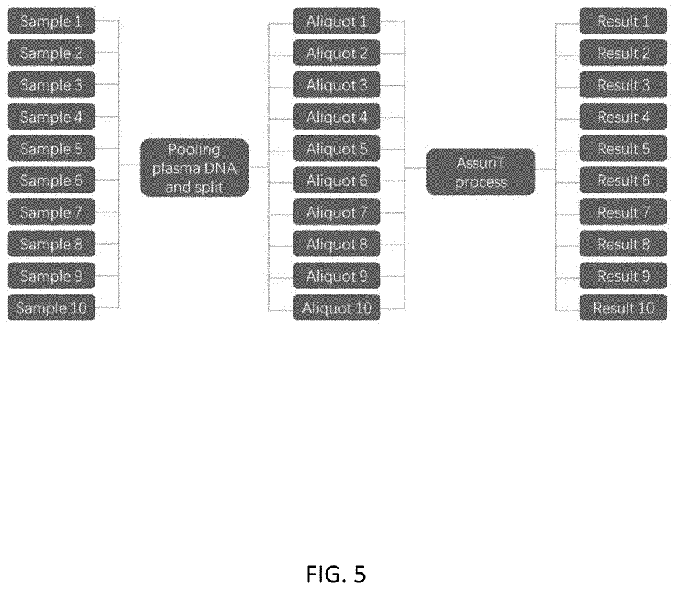

10 plasma-DNA samples were pooled together, then split into 10 aliquots for PCR amplification (FIG. 5). PCR bias correction was conducted as described in Example 1 with data for each aliquot processed separately, obtaining 10 individual sequencing results. Steps 1-4 of Example 1 were carried out followed by calculating the difference of amplicon GC content between each T/R pair (T denotes a locus in the test region, R denotes a locus in the reference region), obtaining an array named Diff_{amplicon GC}, and fitting the logarithmic normalized ratio of amplicon coverage (obtained in step 4 of Example 1) and Diff_{amplicon GC} using robust linear regression:

log(Normalized Ratio of Amplicon Cov.)=β×Diff_{Amplicon GC}+α+ε
where α denotes intercept, β denotes slope and ε denotes residual.

As mentioned above, we generated 10 replicates from the same DNA source. The existence of PCR-bias, i.e., the variation of loci coverage between replicates, is correlated with chemical attributes of loci (GC content, amplicon length, 3′-end stability etc.). The predicted logarithmic normalized ratio of amplicon coverage was obtained using regression parameters acquired by step 6. Next, the difference between observation values and prediction values (which had been corrected for amplification bias) were calculated. FIGS. 4A and 4B show the results of the PCR bias correction. Only one replicate was used to generate the data shown in FIGS. 4A and 4B, but other replicates presented a similar trend. FIG. 4A shows the logarithmic normalized ratio of amplicon coverage before and after PCR bias correction for differences in amplicon GC content. FIG. 4A (left) shows a plot of the data using Diff_{amplicon GC} as the X-axis and the logarithmic normalized ratio of amplicon coverage as the Y-axis, each data point representing a unique T/R pair. The color of each data point depends on the loci in the test region of the corresponding T/R pair: light gray represents chromosome 13; medium gray represents chromosome 18; and dark gray represents chromosome 21. Adding the regression line (the gray line), as calculated according to step 6 of Example 1, demonstrates the correlation between amplicon GC content and normalized loci coverage. FIG. 4 (at right) is similar except for using the residual c as the Y-axis. Diff_{amplicon GC} is not correlated to the residual c, which indicates that the PCR-bias resulting from the difference of amplicon GC content was suppressed. FIG. 4B shows a boxplot instead to illustrate the effectiveness of PCR-bias correction in a more intuitive way. Each box represents a chromosome, under ideal conditions, the median of a box should be zero. However, because of the existence of PCR-bias, the box representing chromosome 21 goes down before correction, which may lead to wrong identification. After PCR-bias correction, the box representing chromosome 21 goes up, demonstrating that the correction was effective.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

1. A method for correcting amplification bias:
a) amplifying target nucleic acids;
b) acquiring amplicon coverage data for the target nucleic acids;
c) calculating a ratio of amplicon coverage between a test genomic region and a reference genomic region for each target nucleic acid;
d) removing outliers;
e) normalizing the ratio of amplieon coverage between the test genomic region and the reference genomic region for each target nucleic acid according to the formula:
$$normalized ratio = \frac{original ratio}{median ( original ratio )} ;$$
f) calculating differences between the test genomic region and the reference genomic region for primer 3′-end stability (Diff_{3′-end stability}). primer melting temperature (Diff_{Tm}), amplicon length (Diff_{amplicon length}), amplicon GC content (Diff_{Amplicon GC}), and GC content of amplicon flanking sequences (Diff_{Amplicon flank GC});
g) fitting data to obtain regression parameter values A₁, A₂, A₃, A₄ and A₅ according to the formula:
log(normalized ratio of amplicon coverage)=A₁×Diff_{3′-end stability}+A₂×Diff_{Tm}+A₃×Diff_{amplicon length}+A₄×Diff_{amplicon GC}+A₅×Diff_{amplicon flank GC}; and
h) correcting amplification bias by using the regression parameter values A₁, A₂, A₃, A₄ and A₅ to calculate a predicted logarithmic normalized ratio of amplicon coverage.

2. The method of claim 1, wherein the target nucleic acids are genomic DNA or RNA.

3. The method of claim 1, wherein said amplifying comprises performing multiplex polymerase chain reaction (PCR).

4. The method of claim 1, wherein said amplifying comprises performing multiplex reverse transcriptase polymerase chain reaction (RT-PCR).

5. The method of claim 1, wherein said target nucleic acids are provided in a plurality of samples.

6. The method of claim 5, further comprising ordering the amplicon coverage data in a matrix as shown in FIG. 1, wherein each row corresponds to a separate amplicon and each column corresponds to a separate sample.

7. The method of claim 6, further comprising creating a ratio matrix of amplicon coverage as shown in FIG. 2.

8. The method of claim 7, further comprising creating a normalized ratio matrix of amplicon coverage with row median as shown in FIG. 3.

9. The method of claim 1, further comprising detecting copy number variation of at least one target nucleic acid after said correcting amplification bias.

10. The method of claim 1, further comprising detecting chromosomal aneuploidy after said correcting amplification bias.

11. (canceled)

12. (canceled)

13. (canceled)

14. The method of claim 1, wherein said target nucleic acids are from a cell, a population of cells, a tissue, a virus, an artificial cell, or a cell-free system.

15. (canceled)

16. The method of claim 1, wherein the amplicon flanking sequences are up to 200 base pairs in length.

17. A computer implemented method for correcting amplification bias, the computer performing steps comprising:
a) receiving inputted amplicon coverage data for a plurality of target nucleic acids;
b) calculating a ratio of amplicon coverage between a test genomic region and a reference genomic region for each target nucleic acid;
c) removing outliers;
d) normalizing the, ratio of amplicon coverage between the test genomic region and the reference genomic region for each target nucleic acid according to the formula:
$$normalized ratio = \frac{original ratio}{median ( original ratio )} ;$$
e) calculating differences between the test genomic region and the reference genomic region for primer 3′-end stability (Diff_{3′-end stability}), primer melting temperature (Diff_{Tm}), amplicon length (Diff_{amplicon length}), amplicon GC content (Diff_{Amplicon GC}), and GC content of amplicon flanking sequences (Diff_{Amiplicon, flank GC});
f) fitting data to obtain regression parameter values A₁, A₂, A₃, A₄ and A₅ according to the formula:
log(normalized ratio of amplicon coverage)=A₁×Diff_{3′-end stability}+A₂×Diff_{Tm}+A₃×Diff_{amplicon length}+A₄×Diff_{amplicon GC}+A₅×Diff_{Amplicon flank GC};
g) correcting amplification bias by using the regression parameter values A₁, A₂, A₃, A₄ and A₅to calculate a predicted logarithmic normalized ratio of amplicon coverage; and
h) displaying information regarding the predicted amplicon coverage with amplification bias correction.

18. The computer implemented method of claim 17, wherein said amplicon coverage data is for target nucleic acids from a plurality of samples.

19. The computer implemented method of claim 18, further comprising, ordering the amplicon coverage data in a matrix as shown in FIG. 1, wherein each row corresponds to a separate amplicon and each column corresponds to a separate sample.

20. The computer implemented method of claim 19, further comprising creating a ratio matrix of amplicon coverage as shown in FIG. 2.

21. The computer implemented method of claim 20, further comprising creating a normalized ratio matrix of amplicon coverage with row median as shown in FIG. 3.

22. The computer implemented method of claim 17, further comprising detecting copy number variation of at least one target nucleic acid atter said correcting amplification bias.

23. The computer implemented method of claim 17, further comprising detecting chromosomal aneuploidy after said correcting amplification bias.

24. A system for correcting amplification bias using the computer implemented method of claim 17 comprising:
a) a storage component for storing amplicon coverage data, wherein the storage component has instructions for correcting the amplification bias stored therein;
b) a computer processor for processing data, wherein the computer processor is coupled to the storage component and configured to execute the instructions stored in the storage component in order to receive amplicon coverage data and correct the amplification bias in the data according to the method of claim 17; and
c) a display component for displaying information regarding the predicted amplicon coverage with amplification bias correction.
